SEQUENCE LISTING (6 sequences)

SEQ ID NO: 1 - DNA, 12 bp, Artificial Sequence
  Example Polynucleotide
  misc_feature (5)..(5): n = 5hmC
  misc_feature (10)..(10): n = 5mC
ccgtnggacngc

SEQ ID NO: 2 - DNA, 12 bp, Artificial Sequence
  Protected Example Polynucleotide
  misc_feature (5)..(5): n = 5hT
ccgtnggactgc

SEQ ID NO: 3 - DNA, 12 bp, Artificial Sequence
  PCR amplified protected example polynucleotide
ccgttggactgc

SEQ ID NO: 4 - DNA, 12 bp, Artificial Sequence
  amplified without protection and deamination example polynucleotide
ccgtcggaccgc

SEQ ID NO: 5 - DNA, 12 bp, Artificial Sequence
  protected example polynucleotide
  misc_feature (1)..(2): n = protected C
  misc_feature (5)..(5): n = 5hmC
  misc_feature (9)..(9): n = protected C
  misc_feature (10)..(10): n = 5mC
  misc_feature (12)..(12): n = protected C
nngtngganngn

SEQ ID NO: 6 - DNA, 12 bp, Artificial Sequence
  converted example polynucleotide
ccgtuggacugc

(19) United States
(12) Patent Application Publication
(10) Pub. No.: US 2022/0290234 A1
(43) Pub. Date: Sep. 15, 2022

(54) DETECTING METHYLCYTOSINE AND ITS DERIVATIVES USING S-ADENOSYL-L-METHIONINE ANALOGS (xSAMS)

(71) Applicants: ILLUMINA, INC., San Diego, CA (US); ILLUMINA CAMBRIDGE LIMITED, Cambridge (GB)

(72) Inventors: Sarah Shultzaberger, San Diego, CA (US); Xiaolin Wu, Cambridge (GB); Eric Brustad, San Diego, CA (US); Niall Gormley, Little Chesterford (GB)

(73) Assignees: ILLUMINA, INC., San Diego, CA (US); ILLUMINA CAMBRIDGE LIMITED, Great Abington (GB)

(21) Appl. No.: 17/694,404

(22) Filed: Mar. 14, 2022

Related U.S. Application Data
(60) Provisional application No. 63/161,330, filed on Mar. 15, 2021.

(51) Int. Cl. C12Q 1/6876; C12Q 1/6806; C07H 19/16

(52) U.S. Cl. CPC C12Q 1/6876; C12Q 1/6806; C12Y 201/01037; C12Y 204/01027; C12Y 302/02029; C12Y 305/04005; C07H 19/16; C12Q 2600/154

(57) ABSTRACT

Examples provided herein are related to detecting methylcytosine and its derivatives using S-adenosyl-L-methionine analogs (xSAMs). Compositions and methods for performing such detection are disclosed. A target polynucleotide may include cytosine (C) and methylcytosine (mC). The method may include (a) protecting the C in the target polynucleotide from deamination; and (b) after step (a), deaminating the mC in the target polynucleotide to form thymine (T). Protecting the C from deamination may include adding a protective group to the 5 position of the C, e.g., using a methyltransferase enzyme that adds the first protective group from an xSAM.

### CROSS-REFERENCE TO RELATED APPLICATION

This application claims the benefit of U.S. Provisional Patent Application No. 63/161,330, filed on Mar. 15, 2021 and entitled “DETECTING METHYLCYTOSINE AND ITS DERIVATIVES USING S-ADENOSYL-L-METHIONINE ANALOGS (xSAMS),” the entire contents of which are incorporated by reference herein.

### FIELD

This application relates to compositions and methods for detecting methylcytosine.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 8549102500_SL.txt. The text file is 2.06 KB, was created on Mar. 9, 2022, and is being submitted electronically via EFS-Web.

### BACKGROUND

Within living organisms, such as humans, selected cytosines (Cs) in the genome may become methylated. For example, S-adenosyl-L-methionine (SAM) is known to be a ubiquitous methyl donor for a variety of biological methylation reactions that are catalyzed by enzymes referred to as methyltransferases (MTases). The enzyme 5-MTase may add a methyl group to the 5-position of cytosine to form 5-methylcytosine (5mC) in a manner such as described in Deen et al., “Methyltransferase-directed labeling of biomolecules and its applications,” Angewandte Chemie International Edition 56: 5182-5200 (2017), the entire contents of which are incorporated by reference herein. Another enzyme may oxidize the cytosine's methyl group to form the 5mC derivative 5-hydroxymethyl cytosine (5hmC), and may oxidize the 5hmC further to form the 5mC derivatives 5-formyl cytosine (5fC) and 5-carboxy cytosine (5caC).

5mC and 5hmC may be referred to as epigenetic markers, and it can be desirable to detect them in a genomic sequence. The current golden standard method for detecting 5mC and 5hmC is bisulfite sequencing, which converts any unmethylated C in the sequence to uracil (U), but does not convert 5mC or 5hmC to the corresponding uracil derivatives. When the sequence is amplified using polymerase chain reaction (PCR), the uracil is amplified as thymidine (T), and as such the unmethylated C is sequenced as T. In comparison, the 5mC and 5hmC are amplified as C, and as such are sequenced as C. Thus, any Cs in the sequence may be identified as corresponding to 5mC or 5hmC because they had not been converted to U. Such a scheme may be referred to as a “three-base” sequencing scheme because any unmethylated C is converted to T. However, this type of scheme reduces sequence complexity and may lead to reduced sequencing quality, lower mapping rates, and relatively uneven coverage of the sequence.

### SUMMARY

Examples provided herein are related to detecting methylcytosine and its derivatives using S-adenosyl-L-methionine analogs (xSAMs). Compositions and methods for performing such detection are disclosed.

Some examples herein provide a method of modifying a target polynucleotide. The target polynucleotide may include cytosine (C) and methylcytosine (mC). The method may include (a) protecting the C in the target polynucleotide from deamination. The method may include (b) after step (a), deaminating the mC in the target polynucleotide to form thymine (T).

In some examples, protecting the C from deamination includes adding a first protective group to the 5 position of the C. In some examples, a first methyltransferase enzyme adds the first protective group to the 5 position of the C. In some examples, the first methyltransferase enzyme adds the first protective group from an S-adenosyl-L-methionine analog (xSAM) having the structure:

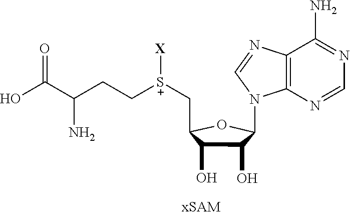

where X includes the first protective group and a methylene group via which the first protective group is coupled to the sulfonium ion (S+).

In some examples, the first methyltransferase enzyme is selected from the group consisting of: DNMT1, DNMT3A, DNMT3B, dam, and CpG (M.SssI).

In some examples, the first protective group includes an alkyne group, a carboxyl group, an amino group, a hydroxymethyl group, an isopropyl group, or a dye.

In some examples, the methyl group of mC inhibits addition of X to the 5 position of the mC.

In some examples, a cytidine deaminase enzyme deaminates the mC. In some examples, X fits within the first methyltransferase enzyme and inhibits activity of the cytidine deaminase enzyme. In some examples, the cytidine deaminase enzyme includes APOBEC. In some examples, the APOBEC is selected from the group consisting of: APOBEC1, APOBEC2, APOBEC3A, APOBEC3B, APOBEC3C, APOBEC3E, APOBEC3F, APOBEC3G, APOBEC3H, and APOBEC4.

In some examples, the target polynucleotide further includes hydroxymethylcytosine (hmC), and step (b) includes deaminating the hmC in the target polynucleotide to form hydroxythymine (hT).

In some examples, the target polynucleotide further includes hydroxymethylcytosine (hmC). The method further may include (c) before step (b), protecting the hmC in the target polynucleotide from deamination. In some examples, step (c) is performed after step (a). In some examples, protecting the hmC from deamination includes adding a second protective group to the hydroxymethyl group of the hmC. In some examples, an enzyme adds the second protective group to the hydroxymethyl group of the hmC. In some examples, the enzyme is selected from the group consisting of: β-glucosyltransferase (βGT) and β-arabinosyltransferase (βAT). In some examples, the second protective group includes a sugar.

In some examples, the method includes performing steps (a) and (b) on a first sample including the target polynucleotide, and performing steps (a), (b), and (c) on a second sample including the target polynucleotide.

In some examples, the target polynucleotide further includes formylcytosine (fC), wherein the formyl group of the fC inhibits deamination of the fC during step (b).

In some examples, the target polynucleotide further includes formylcytosine (fC), and the method further may include (d) before step (b), converting the fC to an unprotected C that is deaminated during step (b) to form uracil (U). In some examples, a thymine deglycosylase enzyme replaces the base of fC with C.

In some examples, the method includes performing steps (a) and (b) on a first sample including the target polynucleotide, and performing steps (a), (b), and (d) on a third sample including the target polynucleotide.

In some examples, the target polynucleotide further includes carboxylcytosine (caC), wherein the carboxyl group of the caC inhibits deamination of the fC during step (b).

In some examples, the target polynucleotide further includes carboxylcytosine (caC), and the method further includes (e) before step (b), converting the caC to unprotected C that is deaminated during step (b) to form uracil (U). In some examples, a third methyltransferase enzyme removes the carboxyl group from caC. In some examples, a thymine deglycosylase enzyme replaces the base of caC with C.

In some examples, the method includes performing steps (a) and (b) on a first sample including the target polynucleotide, and performing steps (a), (b), and (e) on a fourth sample including the target polynucleotide. In some examples, the third sample is the fourth sample, and the second methyltransferase is the third methyltransferase.

In some examples, the target polynucleotide includes DNA.

In some examples, the target polynucleotide includes first and second adapters. In some examples, the first and second adapters are added to the target polynucleotide before step (a). In some examples, the first and second adapters are added to the target polynucleotide after step (b).

Some examples herein provide a method of sequencing a target polynucleotide. The method may include modifying the target polynucleotide in accordance with any of the foregoing methods. The method may include generating a first amplicon of the modified target nucleotide. The first amplicon may include a first guanine (G) at a location complementary to the protected C, and a first adenine (A) at a location complementary to the T. The method may include generating a second amplicon of the first amplicon, the second amplicon including a first unprotected C at a location complementary to the first G, and a first thymine (T) at a location complementary to the first A. The method may include sequencing the first amplicon, the second amplicon, or both the first amplicon and the second amplicon. The method may include identifying the mC based on the first A in the first amplicon, the first T in the second amplicon, or both the first A in the first amplicon and the first T in the second amplicon.

In some examples, the first amplicon includes a second A at a location complementary to the hT and the second amplicon includes a second T at a location complementary to the second A. The method further may include identifying the hmC based on the second A in the first amplicon, the second T in the second amplicon, or both the second A in the first amplicon and the second T in the second amplicon.

In some examples, the first amplicon includes a second G at a location complementary to the hmC and the second amplicon includes a second unprotected C at a location complementary to the second G. The method further may include identifying the hmC based on the second G in the first amplicon, the second unprotected C in the second amplicon, or both the second G in the first amplicon and the second unprotected C in the second amplicon.

In some examples, the first amplicon includes a third G at a location complementary to the fC and the second amplicon includes a third unprotected C at a location complementary to the third G. The method further may include identifying the fC based on the third G in the first amplicon, the third unprotected C in the second amplicon, or both the third G in the first amplicon and the third unprotected C in the second amplicon.

In some examples, the first amplicon includes a third A at a location complementary to the U and the second amplicon includes a third T at a location complementary to the third A. The method further may include identifying the fC based on the third A in the first amplicon, the third T in the second amplicon, or both the third A in the first amplicon and the third T in the second amplicon.

In some examples, the first amplicon includes a fourth G at a location complementary to the caC and the second amplicon includes a fourth unprotected C at a location complementary to the fourth G. The method further may include identifying the caC based on the fourth G in the first amplicon, the fourth unprotected C in the second amplicon, or both the fourth G in the first amplicon and the fourth unprotected C in the second amplicon.

In some examples, the first amplicon includes a fourth A at a location complementary to the U and the second amplicon includes a fourth T at a location complementary to the fourth A. The method further may include identifying the caC based on the fourth A in the first amplicon, the fourth T in the second amplicon, or both the fourth A in the first amplicon and the fourth T in the second amplicon.

Some examples herein provide an isolated polynucleotide from an extracellular fluid sample. The polynucleotide may include cytosine (C) including a protective group at the 5 position; and thymine (T).

In some examples, the first protective group includes an alkyne group, a carboxyl group, an amino group, a hydroxymethyl group, an isopropyl group, or a dye.

In some examples, the polynucleotide includes hydroxymethylcytosine (hmC). In some examples, the hmC includes a second protective group. In some examples, the second protective group includes a sugar.

In some examples, the polynucleotide includes hydroxythymine (hT).

In some examples, the polynucleotide includes formylcytosine (fC).

In some examples, the polynucleotide includes carboxylcytosine (caC).

In some examples, the polynucleotide includes uracil (U).

In some examples, the polynucleotide includes DNA.

In some examples, the polynucleotide includes first and second adapters.

Some examples herein provide an S-adenosyl-L-methionine analog (xSAM) having the structure:

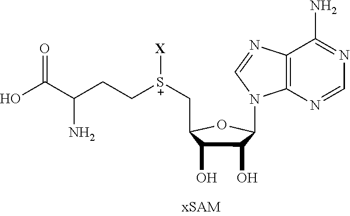

where X includes a protective group and a methylene group via which the protective group is coupled to the sulfonium ion (S+).

In some examples, the protective group includes an alkyne group, a carboxyl group, an amino group, a hydroxymethyl group, an isopropyl group, or a dye.

Some examples herein provide a composition including a polynucleotide, any of the foregoing xSAMs, and a methyltransferase enzyme adding the protective group of the xSAM to cytosine in the polynucleotide.

Some examples herein provide a composition including an isolated polynucleotide and a cytidine deaminase enzyme in an extracellular fluid. The polynucleotide may include (i) cytosine (C) including a protective group at the 5 position, and (ii) methylcytosine (mC) or hydroxymethylcytosine (hmC). The cytidine deaminase enzyme may deaminate the mC to form thymine (T) or deaminating the hmC to form hydroxythymine (hT).

Some examples herein provide a composition including an isolated polynucleotide and a methyltransferase enzyme in an extracellular fluid. The polynucleotide may include (i) cytosine (C) including a protective group at the 5 position, and (ii) formylcytosine (fC) or carboxylcytosine (caC). The composition may include an enzyme converting the fC or caC to C.

Some examples herein provide an isolated polynucleotide and a β-glucosyltransferase (βGT) or β-arabinosyltransferase (βAT) enzyme in an extracellular fluid. The polynucleotide may include (i) cytosine (C) including a first protective group at the 5 position, and (ii) hydroxymethylcytosine (hmC). The βGT or βAT enzyme may add a second protective group to the hmC.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a set of reactions for detecting methylcytosine and its derivatives using S-adenosyl-L-methionine analogs (xSAMs).

FIG. 2 schematically illustrates selected reactions of FIG. 1.

FIG. 3 schematically illustrates additional sets of reaction schemes for detecting methylcytosine and its derivatives, and for distinguishing methylcytosine derivatives from one another, using xSAMs.

### DETAILED DESCRIPTION

Examples provided herein are related to detecting methylcytosine and its derivatives using S-adenosyl-L-methionine analogs (xSAMs). Compositions and methods for performing such detection are disclosed.

As provided herein, a protective group (X) is added to the 5-position of any unmethylated cytosine (C) in a polynucleotide sequence, so as to generate XC which is relatively stable against further reactions that are used to convert any methylcytosine (mC) to thymine (T), and to convert any hydroxymethylcytosine (hmC) to hydroxythymine (hT). When the sequence is amplified using polymerase chain reaction (PCR), the T and hT are amplified as thymine (T), and as such the mC and its derivative hmC are sequenced as T. In comparison, the unmethylated C is amplified, and sequenced, as C. Thus, any Cs in the sequence may be identified as corresponding to C because they had not been converted to T. Such a scheme may be referred to as a “four-base” sequencing scheme because any unmethylated C is sequenced as C. In comparison to a “three-base” sequencing scheme, the present scheme maintains sequence complexity and may lead to enhanced sequencing quality, higher mapping rates, and relatively even coverage of the sequence. Additional reactions are provided for distinguishing mC and its derivatives from one another, thus providing additional analytical tools for characterizing any epigenetic markers in a genomic sequence.

First, some terms used herein will be briefly explained. Then, some example compositions and example methods for detecting methylcytosine and its derivatives using xSAMS will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” refers to a polynucleotide to which nucleotides may be added via a free 3′ OH group. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. A primer may be coupled to a substrate.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, Calif.).

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

As used herein, the term “methylcytosine” or “mC” refers to cytosine that includes a methyl group (—CH₃ or -Me). The methyl group may be located at the 5 position of the cytosine, in which case the mC may be referred to as 5mC.

As used herein, a “derivative” of methylcytosine refers to methylcytosine having an oxidized methyl group. A nonlimiting example of an oxidized methyl group is hydroxymethyl (—CH₂OH), in which case the mC derivative may be referred to as hydroxymethylcytosine or hmC. Another nonlimiting example of an oxidized methyl group is formyl group (—CHO) in which case the mC derivative may be referred to as formylcytosine or fC. Another nonlimiting example of an oxidized methyl group is carboxyl (—COOH), in which case the mC derivative may be referred to as carboxylcytosine or caC. The oxidized methyl group may be located at the 5 position of the cytosine, in which case the hmC may be referred to as 5hmC, the fC may be referred to as 5fC, or the caC may be referred to as 5caC.

As used herein, a “derivative” of thymine (T) refers to thymine having an oxidized methyl group. A nonlimiting example of an oxidized methyl group is hydroxymethyl (—COH), in which case the T derivative may be referred to as hydroxythymine or hT. The oxidized methyl group may be located at the 5 position of the thymine, in which case the hT may be referred to as 5hT.

As used herein, S-adenosyl-L-methionine (SAM) refers to a compound having the structure:

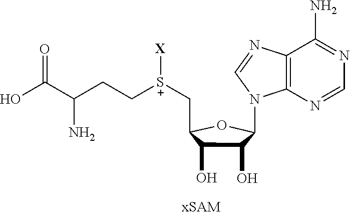

The methyl group bound at the sulfonium (S+) ion may be transferred to cytosine by a methyltransferase in a manner such as described in Deen et al., referenced above. A counterion will likely be present, such as chlorine (Cl−), or the proton may be removed from the COOH to provide a neutral atom. Additionally, the amino acid in solution may be in the zwitterionic isoform (COO−, NH3+).

As used herein, the term S-adenosyl-L-methionine analog (xSAM) refers to a compound having the structure:

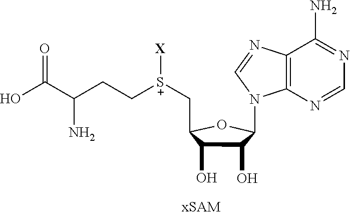

where X includes a protective group and a methylene group via which the protective group is coupled to the S. X may be compatible with the activity of one or more enzymes, and may inhibit the activity of one or more enzymes. For example, as described in greater detail herein, X may be compatible with the activity of a methyltransferase enzyme, such that the methyltransferase may act upon the xSAM to transfer X, which is bound at the sulfonium ion of xSAM, to cytosine to form XC in a similar manner as described in Deem et al., in which the methyltransferase acts upon SAM to transfer the sulfonium-bound methyl group to cytosine to form mC. Additionally, or alternatively, X may be incompatible with the activity of a cytidine deaminase enzyme, such that the cytidine deaminase enzyme may not act upon XC to deaminate the XC in a similar manner as the cytidine deaminase otherwise would act upon C to form U, upon mC to form T, or upon hmC to form hT. Nonlimiting examples of X include a methylenealkyne group

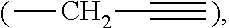

a methylenecarboxyl group

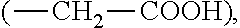

a methyleneamino group (—CH₂—NH₂), a methylenehydroxymethyl group

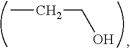

a methyleneisopropyl group

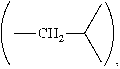

or a methylene dye group (—CH₂-Dye).

As used herein, a “methyltransferase enzyme” or “MTase” refers to an enzyme that may add a methyl group to (or “methylate”) a substrate, or may remove a methyl group from (or “demethylate”) a substrate. Some methyltransferases may add the methyl group (Me) from SAM to a substrate, such as C, and also, or alternatively, may add the protective group (X) from XSAM to such substrate, such as C. Nonlimiting examples of methyltransferases suitable for adding protective group X from XSAM to C include mammalian methyltransferases such as DNMT1, DNMT3A, and DNMT3B described in Jin et al., “DNA methytransferases (DNMTs), DNA damage repair, and cancer,” Adv Exp Med Biol. 754: 3-29 (2013), the entire contents of which are incorporated by reference herein, and bacterial methytransferases such as dam and CpG (M.SssI) commercially available from New England Biolabs (Ipswitch, Mass.). Some methyltransferases may remove an oxidized methyl group (such as formyl or carboxyl) from a substrate, such as caC. Nonlimiting examples of methyltransferases that may decarboxylate caC, in the absence of SAM, include the bacterial C5-methyltransferases M. HhaI and M. SssI (the latter of which also can be used to add the protective group X from XSAM to C in a manner such as described above). For further details of use of a methyltransferase to remove a carboxyl group from caC to form C, see Liutkeviciute et al., “Direct decarboxylation of 5-carboxylcytosine by DNA C5-methyltransferases,” J. Am. Chem. Soc. 136(16): 5884-5887 (2014), the entire contents of which are incorporated by reference herein.

As used herein, “thymine deglycoslyase” (TDG) refers to an enzyme that excises the base from fC or caC and replaces the excised base with C, a reaction that may be referred to as base excision repair (BER). For further details regarding TDG and BER, see Kohli et al., “TET enzymes, TDG and the dynamics of DNA methylation,” Nature 502(7472): 472-479 (2013), the entire contents of which are incorporated by reference herein.

As used herein, a “cytidine deaminase enzyme” refers to an enzyme that deaminates cytosine and/or one or more cytosine derivatives. The deamination may be performed at the 6 position of the cytosine or cytosine derivative. For example, a cytidine deaminase enzyme may deaminate cytosine to form U, may deaminate mC to form T, and/or may deaminate hmC to form hT. A cytidine deaminase enzyme may not necessarily deaminate all possible cytosine derivatives. For example, a cytidine deaminase enzyme may not deaminate cytosine that includes X at the five position, may not deaminate fC to form formyluridine (fU), and/or may not deaminate caC to form carboxyuridine (caU). A nonlimiting example of a cytidine deaminase enzyme that may deaminate cytosine to form U, may deaminate mC to form T, and/or may deaminate hmC to form hT, and that may not deaminate fC to form fU and/or may not deaminate caC to form caU is apolipoprotein B mRNA editing enzyme, catalytic polypeptide-like (APOBEC). Nonlimiting examples of such APOBECs include APOBEC1, APOBEC2, APOBEC3A, APOBEC3B, APOBEC3C, APOBEC3E, APOBEC3F, APOBEC3G, APOBEC3H, and APOBEC4.

As used herein, a “β-glucosyltransferase enzyme” or “βGT” refers to an enzyme that adds a glucose group (e.g., glucose or a glucose derivative) to hmC, for example to the hydroxymethyl group at the 5 position of the hmC to form β-glucosyl-5-hydroxymethyl cytosine (1,2). A nonlimiting example of a βGT is the T4 phage β-glucosyltransferase (T-4BGT), commercially available from New England Biolabs (Ipswitch, Mass.).

As used herein a “β-arabinosyltransferase enzyme” or “βAT” refers to an enzyme that adds an arabinose group to hmC, for example to the hydroxymethyl group at the 5 position of the hmC to form arabinosyl-hmC. A nonlimiting example of a βAT is the T4-like phage RB69 ORF003c described in Thomas et al., “The odd ‘RB’ Phage—identification of arabinosylation as a new epigenetic modification of DNA in T4-like phage RB69,” Viruses 10(6): 313, 18 pages (2018), the entire contents of which are incorporated by reference herein.

As used herein, a “protective group” is intended to mean a chemical group that inhibits the activity of an enzyme. For example, a protective group that is coupled via a methylene group to the 5-position of cytosine may inhibit activity of a cytidine deaminase enzyme that otherwise would deaminate the cytosine to form uracil. As another example, a protective group (e.g., a sugar such as glucose or arabinose) at the hydroxymethyl group at the 5 position of hmC may inhibit activity of a cytidine deaminase enzyme that otherwise would deaminate the hmC to form hydroxythymine.

Compositions and Methods for Detecting Methylcytosine and its Derivatives Using xSAMS

Some examples provided herein are related to detecting methylcytosine and its derivatives using xSAMs. Compositions and methods for performing such detection are disclosed.

For example, a target polynucleotide having a sequence that includes cytosine (C) and methylcytosine (mC), and also may include hydroxymethylcytosine (hmC) may be modified in such a manner as to protect the C from deamination, and then deaminating the mC to form thymine (T) and deaminating the hmC to form hydroxythymine (hT). In a manner such as described in greater detail below, when the sequence subsequently is amplified using polymerase chain reaction (PCR), the T and any hT are amplified as thymidine (T), and as such the mC and hmC may be sequenced as T. In comparison, the unmethylated (and protected) C is amplified, and sequenced, as C. Thus, any Cs in the sequence may be identified as corresponding to C because they had not been converted to T or T derivatives as are mC and hmC. As such, the present methods provide a “four-base” sequencing method in which the unmethylated C may be sequenced as C, and thus preserves the genomic information carried by that base. In a manner such as described in greater detail below, the mC and hmC may be distinguished from one another using an additional reaction scheme.

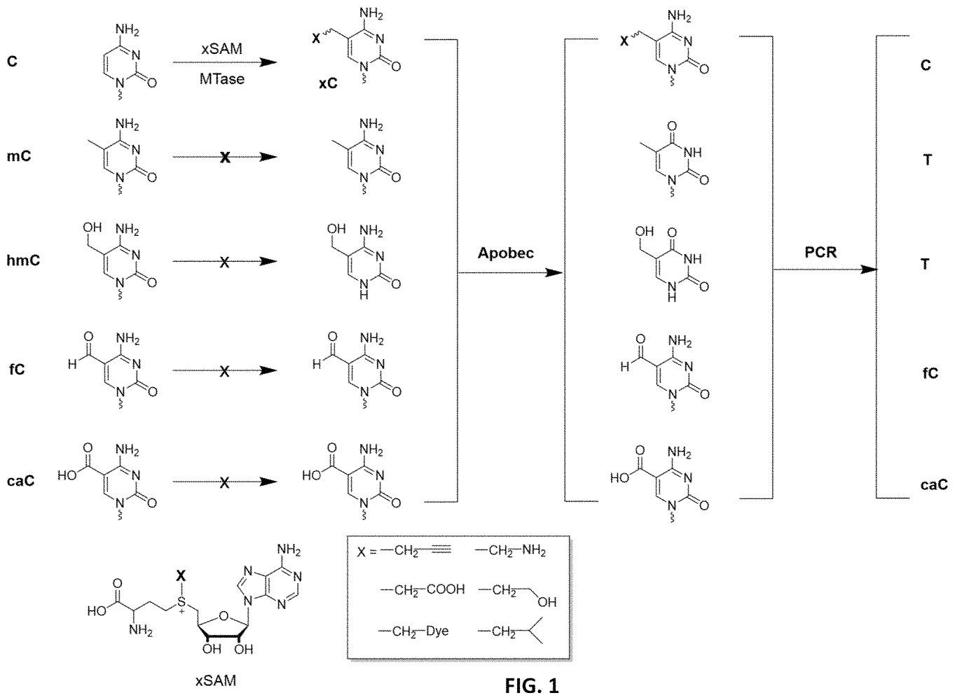

FIG. 1 schematically illustrates a set of reactions for detecting methylcytosine (mC) and its derivatives using xSAMS. As illustrated in FIG. 1, protecting the C from deamination may include adding a first protective group to the 5 position of the C. For example, a first methyltransferase enzyme (MTase) may add X to the 5 position of the C to form XC in a manner such as illustrated in FIG. 1, where X includes a protective group and a methylene group via which the protective group is coupled to the C. Illustratively, the first methyltransferase enzyme may add X from an xSAM having the structure:

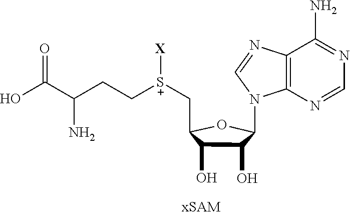

where X includes the first protective group and a methylene group via which the first protective group is coupled to the sulfonium ion. In nonlimiting examples, the first protective group may include an alkyne group, a carboxyl group, an amino group, a hydroxymethyl group, an isopropyl group, or a dye. The xSAM, having a sulfonium-bound first protective group and methylene group, may serve as a surrogate cofactor in place of SAM, having a sulfonium-bound methyl group, and as such the methyltransferase may covalently deposit the methylene group (with the first protective group coupled thereto) at the 5 position of any unmethylated C in the target polynucleotide, forming 5XC. During action of the methyltransferase, a composition may be formed that includes the polynucleotide, the xSAM, and the methyltransferase enzyme adding X from the xSAM to C in the polynucleotide. It will be appreciated that a suitable amount of the methyltransferase and xSAM may be mixed with the polynucleotide in an extracellular liquid. For example, xSAM is a stoichiometric reagent, so at least as much xSAM may be added as there are Cs in a genomic sample, and an excess of xSAM may be added.

Note that methyltransferase may be unable to add X (and thus may be unable to add the first protective group) to any mC and/or any mC derivatives in the target polynucleotide, as illustrated in FIG. 1. For example, the methyl group of mC may inhibit addition of the X (and first protective group) to the 5 position of the mC, because the methyl group already occupies that location. Similarly, the hydroxymethyl group of any hmC may inhibit addition of X (and the first protective group) to the 5 position of the hmC; the formyl group of any fC may inhibit addition of X (and the first protective group) to the 5 position of the fC; and the carboxyl group of any caC may inhibit addition of X (and the first protective group) to the 5 position of the caC.

Following protection of the C in the target polynucleotide, the mC and/or any of its derivatives may be deaminated, e.g., using a cytidine deaminase enzyme. In this regard, although the first protective group may be selected so as fit within the first methyltransferase enzyme and thus may be compatible with activity of the first methyltransferase enzyme, the first protective group may inhibit activity of the cytidine deaminase enzyme. Additionally, the formyl group of any fC may inhibit activity of the cytidine deaminase enzyme, and the carboxyl group of any caC may inhibit activity of the cytidine deaminase enzyme. In comparison, the methyl group of mC and the hydroxymethyl group of hmC may be compatible with activity of the cytidine deaminase enzyme. As such, as illustrated in FIG. 1, XC, any fC, and any caC may not be deaminated by the cytidine deaminase enzyme, while any mC may be deaminated to form T, any hmC may be deaminated to form hT. During action of the cytidine deaminase enzyme, a composition may be formed that includes the polynucleotide and the cytidine deaminase enzyme in an extracellular fluid. The polynucleotide may include XC and mC and/or hmC. The cytidine deaminase enzyme may be deaminating the mC to form T or deaminating the hmC to form hT. It will be appreciated that a suitable amount of the cytidine deaminase enzyme may be mixed with the polynucleotide in the extracellular liquid. For example, cytidine deaminase may be added in catalytic amounts, e.g., less than the number of mCs and hmCs to be deaminated.

As illustrated in FIG. 1, PCR then may be performed to generate amplicons of the target polynucleotide. In a first set of the amplicons, the unmethylated, protected C is amplified as C, while the T and hT are amplified as T, and fC and caC are amplified as C. It will be appreciated that a second set of complementary amplicons also are generated using PCR, in which the unmethylated, protected C is amplified as G, while the T and hT are amplified as A, and fC and caC are amplified as G. The amplicons then may be sequenced using known techniques, such as sequencing-by-synthesis (SBS). The locations in the target polynucleotide at which mC and hmC were located, and at which T and hT were generated using deamination while the C is protected using the present xSAM, may be determined by comparing the sequence of the resulting amplicons to the sequence of amplicons in which the mC and hmC are not deaminated and thus are amplified and sequenced as C (or, in the complementary amplicons, as G). Bases that are T (or A) in the deaminated amplicons and that are C (or G) in the non-deaminated amplicons may be identified as corresponding to hC and/or hmC.

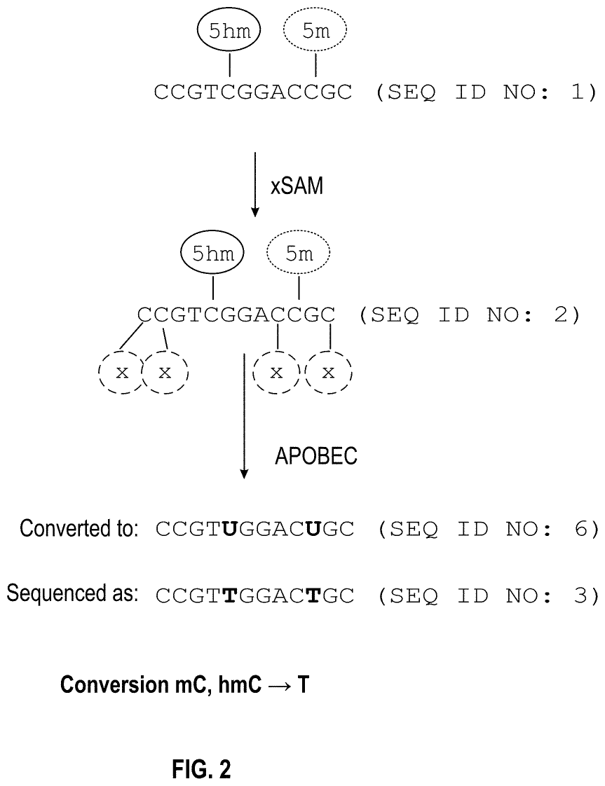

For example, FIG. 2 schematically illustrates selected reactions of FIG. 1. In FIG. 2, an example polynucleotide sequence CCGT(5hmC)GGAC(mC)GC (SEQ ID NO: 1) is shown. The other Cs are protected using a protective group (X) transferred from an xSAM by a methyltransferase enzyme. A cytidine deaminase enzyme, such as APOBEC, then is used to deaminate the 5hmC and 5mC, resulting in the sequence CCGT(5hT)GGAC(T)GC (SEQ ID NO: 2) which is amplified by PCR and then sequenced as CCGTTGGACTGC (SEQ ID NO: 3), where the bolded Ts correspond to 5hmC and mC in the original sequence. The presence and locations in the target polynucleotide of the 5hmC and mC may be detected by also amplifying and sequencing the target polynucleotide without the protection and deamination steps to obtain the sequence CCGTCGGACCGC (SEQ ID NO: 4) where the bolded Cs correspond to 5hmC and mC in the original sequence; and comparing the sequence of those amplicons of the target polynucleotide to that of the sequence of the amplicons following protection and deamination. Through such comparison, it may be seen that the bolded Cs are “converted” from C to T, indicating that deamination occurred and that therefore mC or hmC were originally present at those locations.

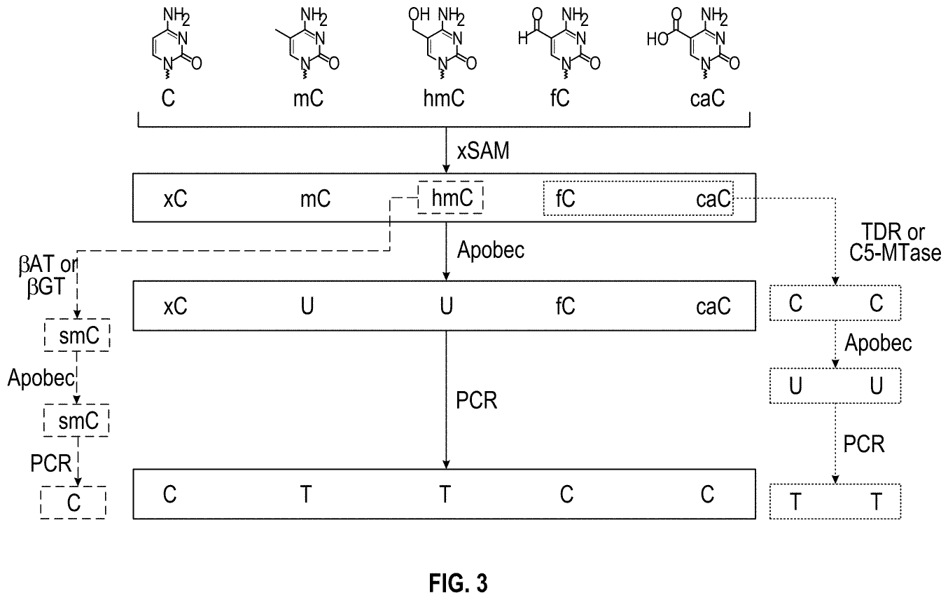

Additionally, as noted further above, the present disclosure provides methods for distinguishing methylcytosine and certain of its derivatives from one another. For example, FIG. 3 schematically illustrates additional sets of reaction schemes for detecting mC and its derivatives, and for distinguishing methylcytosine derivatives from one another, using xSAMS.

As illustrated in FIG. 3, mC and hmC may be distinguished from one another using an additional reaction after protecting the C using xSAMs, but before deamination. Such reaction protects the hmC in the target polynucleotide from deamination, and as such, the hmC is not converted to hT during deamination (and thus is amplified and sequenced as C), while the mC is converted to T (and thus is amplified and sequenced as T). Protecting the hmC from deamination may include adding a second protective group to the hydroxymethyl group of the hmC to form gmC. Illustratively, a sugar-transferring enzyme such as a β-glucosyltransferase (βGT) or β-arabinosyltransferase (βAT) enzyme may add the second protective group to the hydroxymethyl group of the hmC. The second protective group may include a sugar transferred from a sugar donor, such as glucose or glucose derivative transferred from a glucosyl donor (e.g., UDP-glucose, or UDP-6-azide-glucose), or arabinose transferred from an arabinose donor (e.g., UDP-arabinose), forming sugar-methylcytosine (sMC). During action of the sugar transferring enzyme, a composition may be formed that includes the polynucleotide and the enzyme in an extracellular fluid. The polynucleotide may include XC and hmC, and the enzyme may be adding the second protective group to the hmC. It will be appreciated that a suitable amount of the enzyme may be mixed with the polynucleotide in the extracellular liquid. For example, the enzyme may be added in catalytic amounts, while the sugar donor may be added in a stoichiometric amount or in excess.

The unprotected methylcytosine in the polynucleotide then may be deaminated to form T, e.g., using the cytidine deaminase enzyme in a manner such as described with reference to FIG. 1, and the sequence then amplified and sequenced. Note that the use of a glucose derivative such as 6-azide-glucose may allow further modifications to the glucose, e.g., such as via a click chemistry reaction of a dye with the azide in a manner such as described in Song et al., “Simultaneous single-molecule epigenetic imaging of DNA methylation and hydroxymethylation,” PNAS 113(16): 4338-4343 (2016), the entire contents of which are incorporated by reference herein.

So as to distinguish the hmC from mC, the C protection and deamination steps described with reference to FIG. 1 may be performed on a first sample including the target polynucleotide followed by amplification and sequencing; and the C protection, hmC protection, and deamination steps described with reference to FIG. 3 may be performed on a second sample including the target polynucleotide, followed by amplification and sequencing. The sequence of the amplicons from the first sample may be compared to that of the amplicons from the second sample and/or to amplicons of the original sequence. Through such comparisons, it may be understood that Cs that are “converted” from C to T in the first sample, as compared to the original sequence, correspond to mC or hmC; and that such Cs that are not similarly “converted” from T to C in the second sample, as compared to the first sample, correspond to hmC.

Additionally, or alternatively, as illustrated in FIG. 3, fC and caC may be distinguished from C using one or more additional reactions after protecting the C using xSAMs, but before deamination. More specifically, if the target polynucleotide includes fC and/or caC, the formyl group from any fC and/or the carboxyl group from any caC may be removed before deamination to form an unprotected C that may be deaminated to form U. Removal of the carboxyl group may be performed using a methyltransferase enzyme such as described elsewhere herein, or the base of the fC or caC may be replaced with C using thymine deglycosylase (TDG) in a manner such as described elsewhere herein. The unprotected C in the polynucleotide then may be deaminated to form U, e.g., using the cytidine deaminase enzyme in a manner such as described with reference to FIG. 1, and the sequence then amplified and sequenced. So as to distinguish the fC and/or caC from C, the C protection and deamination steps described with reference to FIG. 1 may be performed on a first sample including the target polynucleotide followed by amplification and sequencing; and the C protection, fC and/or caC deprotection, and deamination steps described with reference to FIG. 3 may be performed on a second sample including the target polynucleotide, followed by amplification and sequencing. The sequence of the amplicons from the first sample may be compared to that of the amplicons from the second sample and/or to amplicons of the original sequence. Through such comparisons, it may be understood that Cs that remain C in the first sample, as compared to the original sequence, correspond to C, fC, or caC; and that such Cs that are “converted” from C to T in the second sample, as compared to the first sample, correspond to fC or caC. During action of the methyltransferase or TDG enzyme, a composition may be formed that includes the polynucleotide and the enzyme in an extracellular fluid. The polynucleotide may include XC and fC and/or caC. The enzyme may be converting the fC and/or the caC to C. It will be appreciated that a suitable amount of the methyltransferase enzyme may be mixed with the polynucleotide in the extracellular liquid, e.g., in a catalytic amount.

In some examples provided herein, the target polynucleotide includes DNA, although it will be appreciated that the present methods and compositions may be suitably modified to detect mC and/or its derivatives in any suitable type of polynucleotide, such as RNA. The polynucleotide may be isolated and from an extracellular fluid sample, and may include C including a first protective group at the 5 position; and T such as provided using the reaction schemes described with reference to FIGS. 1-2. The first protective group may be coupled to the C via a methylene group and may include, illustratively, an alkyne group, a carboxyl group, an amino group, a hydroxymethyl group, an isopropyl group, or a dye. The polynucleotide further may include hmC, which may include a second protective group, such as a sugar (e.g., glucose or arabinose), such as provided using the reaction schemes described with reference to FIG. 3. Alternatively, the polynucleotide further may include hT, such as provided using the reaction schemes described with reference to FIGS. 1-2. The polynucleotide further may include formylcytosine (fC) and/or may include carboxylcytosine (caC), such as provided using the reaction schemes described with reference to FIGS. 1-2. Alternatively, the polynucleotide may include U, such as provided using the reaction schemes described with reference to FIG. 3.

So as to facilitate amplification and sequencing, the target polynucleotide may include first and second adapters, e.g., which flank the sequence of interest. Such adapters may be added to the target polynucleotide before protecting the C using xSAMS, may be added to the target polynucleotide after the deamination step, or may be added at any other suitable time.

To provide some additional detail regarding sequencing target polynucleotides which are modified in any suitable manner provided herein, first and second complementary amplicons of the modified target nucleotide may be generated. The first amplicon may include a first C at a location complementary to the protected C (XC), and a first adenine (A) at a location complementary to the T. The second amplicon may include a first unprotected C at a location complementary to the first G, and a first thymine (T) at a location complementary to the first A. The first amplicon, the second amplicon, or both the first amplicon and the second amplicon may be sequenced. The mC may be identified based on the first A in the first amplicon, the first T in the second amplicon, or both the first A in the first amplicon and the first T in the second amplicon, e.g., in a manner such as described with reference to FIGS. 1 and 2.

In some examples such as described with reference to FIGS. 1 and 2, the first amplicon includes a second A at a location complementary to the hT and the second amplicon includes a second T at a location complementary to the second A. The hmC may be identified based on the second A in the first amplicon, the second T in the second amplicon, or both the second A in the first amplicon and the second T in the second amplicon. In other examples such as using the additional reactions described with reference to FIG. 3, the first amplicon includes a second G at a location complementary to the hmC and the second amplicon includes a second unprotected C at a location complementary to the second G. The hmC may be identified based on the second G in the first amplicon, the second unprotected C in the second amplicon, or both the second G in the first amplicon and the second unprotected C in the second amplicon.

In some examples such as described with reference to FIGS. 1 and 2, the first amplicon includes a third G at a location complementary to the fC and the second amplicon includes a third unprotected C at a location complementary to the third G. The fC may be identified based on the third G in the first amplicon, the third unprotected C in the second amplicon, or both the third G in the first amplicon and the third unprotected C in the second amplicon. In other examples such as using the additional reactions described with reference to FIG. 3, the first amplicon includes a third A at a location complementary to the U and the second amplicon includes a third T at a location complementary to the third A. The fC may be identified based on the third A in the first amplicon, the third T in the second amplicon, or both the third A in the first amplicon and the third T in the second amplicon.

In some examples such as described with reference to FIGS. 1 and 2, the first amplicon includes a fourth G at a location complementary to the caC and the second amplicon includes a fourth unprotected C at a location complementary to the fourth G. The caC may be identified based on the fourth G in the first amplicon, the fourth unprotected C in the second amplicon, or both the fourth G in the first amplicon and the fourth unprotected C in the second amplicon. In other examples such as using the additional reactions described with reference to FIG. 3, the first amplicon includes a fourth A at a location complementary to the U and the second amplicon includes a fourth T at a location complementary to the fourth A. The caC may be identified based on the fourth A in the first amplicon, the fourth T in the second amplicon, or both the fourth A in the first amplicon and the fourth T in the second amplicon.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of modifying a target polynucleotide, the target polynucleotide comprising cytosine (C) and methylcytosine (mC), the method comprising:
(a) protecting the C in the target polynucleotide from deamination;
(b) after step (a), deaminating the mC in the target polynucleotide to form thymine (T).

2. The method of claim 1, wherein protecting the C from deamination comprises adding a first protective group to the 5 position of the C.

3. The method of claim 2, wherein a first methyltransferase enzyme adds the first protective group to the 5 position of the C.

4. The method of claim 3, wherein the first methyltransferase enzyme adds the first protective group from an S-adenosyl-L-methionine analog (xSAM) having the structure: 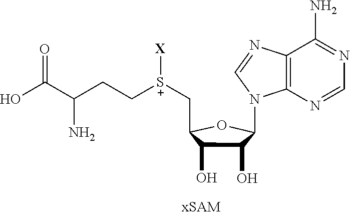
where X includes the first protective group and a methylene group via which the first protective group is coupled to the sulfonium ion (S+).

5. The method of claim 2, wherein the first methyltransferase enzyme is selected from the group consisting of: DNMT1, DNMT3A, DNMT3B, dam, and CpG (M.SssI).

6. The method of claim 2, wherein the first protective group comprises an alkyne group, a carboxyl group, an amino group, a hydroxymethyl group, an isopropyl group, or a dye.

7. The method of claim 2, wherein the methyl group of mC inhibits addition of X to the 5 position of the mC.

8. The method of claim 1, wherein a cytidine deaminase enzyme deaminates the mC.

9. The method of claim 8, wherein X fits within the first methyltransferase enzyme and inhibits activity of the cytidine deaminase enzyme.

10. The method of claim 8, wherein the cytidine deaminase enzyme comprises APOBEC.

11. The method of claim 10, wherein the APOBEC is selected from the group consisting of: APOBEC1, APOBEC2, APOBEC3A, APOBEC3B, APOBEC3C, APOBEC3E, APOBEC3F, APOBEC3G, APOBEC3H, and APOBEC4.

12. The method of claim 1, wherein the target polynucleotide further comprises hydroxymethylcytosine (hmC), and step (b) comprises deaminating the hmC in the target polynucleotide to form hydroxythymine (hT).

13. The method of claim 1, wherein the target polynucleotide further comprises hydroxymethylcytosine (hmC), the method further comprising:
(c) before step (b), protecting the hmC in the target polynucleotide from deamination.

14. The method of claim 13, wherein step (c) is performed after step (a).

15. The method of claim 13, wherein protecting the hmC from deamination comprises adding a second protective group to the hydroxymethyl group of the hmC.

16. The method of claim 15, wherein an enzyme adds the second protective group to the hydroxymethyl group of the hmC.

17. The method of claim 16, wherein the enzyme is selected from the group consisting of: β-glucosyltransferase (βGT) and β-arabinosyltransferase (βAT).

18. The method of claim 15, wherein the second protective group comprises a sugar.

19. The method of claim 13, comprising performing steps (a) and (b) on a first sample including the target polynucleotide, and performing steps (a), (b), and (c) on a second sample including the target polynucleotide.

20. The method of claim 1, wherein the target polynucleotide further comprises formylcytosine (fC), wherein the formyl group of the fC inhibits deamination of the fC during step (b).

21. The method of claim 1, wherein the target polynucleotide further comprises formylcytosine (fC), the method further comprising:
(d) before step (b), converting the fC to an unprotected C that is deaminated during step (b) to form uracil (U).

22. The method of claim 21, wherein a thymine deglycosylase enzyme replaces the base of fC with C.

23. The method of claim 21, comprising performing steps (a) and (b) on a first sample including the target polynucleotide, and performing steps (a), (b), and (d) on a third sample including the target polynucleotide.

24. The method of claim 1, wherein the target polynucleotide further comprises carboxylcytosine (caC), wherein the carboxyl group of the caC inhibits deamination of the fC during step (b).

25. The method of claim 1, wherein the target polynucleotide further comprises carboxylcytosine (caC), the method further comprising:
(e) before step (b), converting the caC to unprotected C that is deaminated during step (b) to form uracil (U).

26. The method of claim 25, wherein a second methyltransferase enzyme removes the carboxyl group from caC.

27. The method of claim 25, wherein a thymine deglycosylase enzyme replaces the base of caC with C.

28. The method of claim 25, comprising performing steps (a) and (b) on a first sample including the target polynucleotide, and performing steps (a), (b), and (e) on a fourth sample including the target polynucleotide.

29. The method of claim 1, wherein the target polynucleotide comprises DNA.

30. The method of claim 1, wherein the target polynucleotide comprises first and second adapters.

31. The method of claim 30, wherein the first and second adapters are added to the target polynucleotide before step (a).

32. The method of claim 30, wherein the first and second adapters are added to the target polynucleotide after step (b).

33.-56. (canceled)
